(19) United States
(12) Patent Application Publication
(10) Pub. No.: US 2010/0158820 A1
(43) Pub. Date: Jun. 24, 2010

(54) XYLITOL DENTAL MAINTENANCE SYSTEM

(76) Inventor: Donald W. Bailey, Provo, UT (US)

Correspondence Address: TERRENCE J. EDWARDS;CLAYTON, HOWARTH & CANNON, P.C., P.O. BOX 1909, SANDY, UT, 84091, US

(21) Appl. No.: 12/648,188

(22) Filed: Dec. 28, 2009

Related U.S. Application Data
(63) Continuation-in-part of application No. 10/873,766, filed on Jun. 21, 2004.
(60) Provisional application No. 60/480,516, filed on Jun. 20, 2003.

(51) Int. Cl. A61K 8/34; A61K 9/68; A61Q 11/00

(52) U.S. Cl. 424/48; 424/49

(57) ABSTRACT

A system, kit and method for promoting oral hygiene are disclosed. The kit may comprise several compositions or a single composition, each of which comprises an effective amount of xylitol. For example, the system and kit may comprise a chewing gum composition, a morsel composition, a paste-like composition, and an oral rinse composition; or any one of the above compositions; or any combination of the above compositions. It will be appreciated that a novel method for using such a system and kit for promoting oral hygiene may include: administering about 0.5 grams to about 2.0 grams of xylitol through the paste-like composition in the morning and in the evening, administering about 0.5 grams to about 2.0 grams of xylitol after consumption of nutrition through chewing the gum composition or through the morsel composition, and administering about 0.5 grams to about 1.5 grams of xylitol per application through the oral rinse composition.

### CROSS-REFERENCE TO RELATED APPLICATIONS

This application is a continuation-in-part of co-pending U.S. patent application Ser. No. 10/873,766, filed Jun. 21, 2004, entitled “XYLITOL DENTAL MAINTENANCE SYSTEM,” which claimed the benefit of U.S. Provisional Application No. 60/480,516, filed Jun. 20, 2003, entitled “XYLITOL DENTAL MAINTENANCE SYSTEM,” both of which are hereby incorporated by reference herein in their entireties, including but not limited to those portions that specifically appear hereinafter, the incorporation by reference being made with the following exception: In the event that any portion of either of the above-referenced applications is inconsistent with this application, this application supercedes said portion of said above-referenced applications.

### STATEMENT REGARDING FEDERALLY SPONSORED RESEARCH OR DEVELOPMENT

Not Applicable.

### BACKGROUND

The disclosure relates generally to oral care compositions and methods of use, and more particularly, but not necessarily entirely, to a dental maintenance system that integrates multiple cavity protecting products into an integrated and easily implemented oral hygiene kit and system.

Dental caries is an ecological disease in which the diet, the host and the microbial flora interact over a period of time in such a way as to encourage demineralization of the tooth enamel with resultant caries formation. Dental caries is one of the most common diseases in the world today, and until recently almost everyone in the world had experienced tooth decay in their lifetime. However, today many people are caries free and there has been a forty to sixty percent (40-60%) reduction in the incidence of tooth decay within the Western world. Most developed countries and many non-industrialized countries are now well below the World Health Organization's goal of less than three decayed, missing or filled teeth per twelve-year old child.

Teeth are composed of a thin layer (1-2 mm) of dental enamel, which forms the hard protective coating over each tooth. Teeth consist mainly of calcium, phosphate and other ions in a structure known as “hydroxyapatite.” Dental enamel is porous and is susceptible to acid dissolution during the process of demineralization. This demineralization process is offset by the repair process known as remineralization. Tooth susceptibility to dental caries varies among individuals. Although the reasons for the variation are not fully known, some influences include: (a) the shape, size and order of the teeth that affect the “washing” effects of saliva, which is largely determined by hereditary factors; (b) salivary components which can be critical in controlling dental caries since salivary components affect bacteria, immune status, plaque formation, and enamel structure and can neutralize acids, and therefore saliva has a vital role in the balance between demineralization and remineralization; and (c) enamel structure can be altered by a selection of mineral ions and fluoride, as well as by acid, and the balance between demineralization and remineralization of the enamel determines whether caries will occur.

Dental caries, or cavities, are bacterial infections that cause the tooth structure to deteriorate. The compositions disclosed herein fight cavities from their novel use of xylitol in several integrated products. Xylitol is a clinically proven cariostatic sweetener that reduces the ability of cavity-causing bacteria to impact oral health. Because of xylitol's cariostatic attributes, cavities cannot form in its presence. As such, it is a valuable tool in preventing and fighting cavities. While the United States Food and Drug Administration has not approved cavity prevention claims for xylitol, several studies substantiate its efficacy. Some studies even suggest that xylitol may aid in the reversal of certain types of cavities.

The features and advantages of the disclosure will be set forth in the description which follows, and in part will be apparent from the description, or may be learned by the practice of the disclosure without undue experimentation. The features and advantages of the disclosure may be realized and obtained by means of the compositions and combinations particularly pointed out herein.

### BRIEF DESCRIPTION OF THE DRAWINGS

The features and advantages of the disclosure will become apparent from a consideration of the subsequent detailed description presented in connection with the accompanying drawings in which:

FIG. 1 is a chart illustrating the change in decayed, missing, and/or filled surfaces (DMFS) of subjects in several groups;

FIG. 2 is a chart illustrating the effect of xylitol on the rehardening of dentine lesions in the primary dentition;

FIG. 3 is a flow chart illustrating an exemplary method of promoting oral hygiene and to reduce dental plaque, suppress bacterial growth, and reduce adhesiveness of plaque in a user's oral cavity to thereby inhibit formation of dental caries in accordance with the teachings and principles of the disclosure;

FIG. 4 is another flow chart illustrating an exemplary method of promoting oral hygiene and to reduce dental plaque, suppress bacterial growth, and reduce adhesiveness of plaque in a user's oral cavity to thereby inhibit formation of dental caries in accordance with the teachings and principles of the disclosure;

FIG. 5 is still another flow chart illustrating an exemplary method of promoting oral hygiene and to reduce dental plaque, suppress bacterial growth, and reduce adhesiveness of plaque in a user's oral cavity to thereby inhibit formation of dental caries in accordance with the teachings and principles of the disclosure; and

FIG. 6 is yet another flow chart illustrating an exemplary method of promoting oral hygiene and to reduce dental plaque, suppress bacterial growth, and reduce adhesiveness of plaque in a user's oral cavity to thereby inhibit formation of dental caries in accordance with the teachings and principles of the disclosure.

FIG. 7 is still another flow chart illustrating an exemplary method of promoting oral hygiene and to reduce dental plaque, suppress bacterial growth, and reduce adhesiveness of plaque in a user's oral cavity to thereby inhibit formation of dental caries in accordance with the teachings and principles of the disclosure.

FIG. 8 is yet another flow chart illustrating an exemplary method of promoting oral hygiene and to reduce dental plaque, suppress bacterial growth, and reduce adhesiveness of plaque in a user's oral cavity to thereby inhibit formation of dental caries in accordance with the teachings and principles of the disclosure.

FIG. 9 is a flow chart illustrating an embodiment of a kit promoting oral hygiene and to reduce dental plaque, suppress bacterial growth, and reduce adhesiveness of plaque in a user's oral cavity to thereby inhibit formation of dental caries in accordance with the teachings and principles of the disclosure.

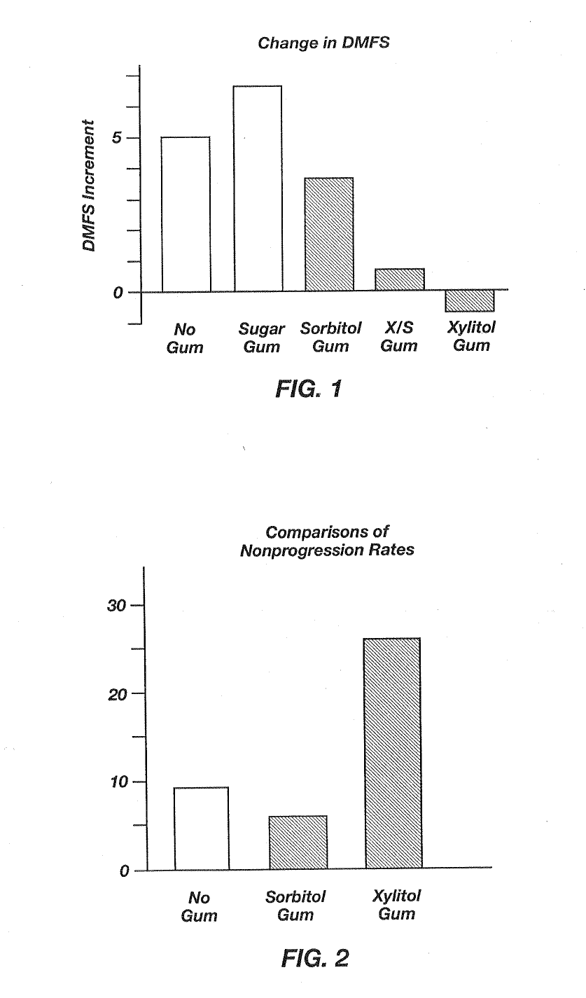

### DETAILED DESCRIPTION

For the purposes of promoting an understanding of the principles in accordance with the disclosure, reference will now be made to the embodiments and examples of the disclosure, and specific language will be used to describe the same. It will nevertheless be understood that no limitation of the scope of the disclosure is thereby intended. Any alterations and further modifications of the inventive features contained herein, and any additional applications of the principles of the disclosure as described herein, which would normally occur to one skilled in the relevant art and having possession of this disclosure, are to be considered within the scope of the disclosure claimed.

Before the system, kit and method for promoting oral hygiene are disclosed and described, it is to be understood that this disclosure is not limited to the particular compositions, configurations, process steps, ingredients and materials disclosed herein as such configurations, process steps, and materials may vary somewhat. It is also to be understood that the terminology employed herein is used for the purpose of describing particular embodiments only and is not intended to be limiting since the scope of the disclosure will be limited only by the appended claims and equivalents thereof.

The publications and other reference materials referred to herein to describe the background of the disclosure, and to provide additional detail regarding the practice of using xylitol, are hereby incorporated by reference herein in their entireties, with the following exception: In the event that any portion of said reference materials is inconsistent with this application, this application supercedes said reference materials. The reference materials discussed herein are provided solely for their disclosure prior to the filing date of this application. Nothing herein is to be construed as a suggestion or admission that the inventors are not entitled to antedate such disclosure by virtue of prior invention, or to distinguish the disclosure from the subject matter disclosed in the reference materials.

It must be noted that, as used in this specification and the appended claims, the singular forms “a,” “an,” and “the” include plural referents unless the context clearly dictates otherwise.

In describing and claiming the disclosure, the following terminology will be used in accordance with the definitions set out below.

As used herein, the terms “comprising,” “including,” “containing,” “characterized by,” and grammatical equivalents thereof are inclusive or open-ended terms that do not exclude additional, unrecited elements or method steps.

As used herein, the phrase “consisting of” and grammatical equivalents thereof exclude any element, step, or ingredient not specified in the claim.

As used herein, an “effective amount” is an amount sufficient to effect beneficial or desired results. An effective amount can be administered in one or more administrations, applications or dosages. For example, an effective amount of xylitol is an amount sufficient to reduce dental plaque, suppress bacterial growth, and reduce adhesiveness of plaque, to thereby inhibit formation of dental caries. Applicant has found that administering about 6 grams to about 10 grams of xylitol in a single day may be effective to reduce dental plaque, suppress bacterial growth, and reduce adhesiveness of plaque, to thereby inhibit formation of dental caries.

Applicant has discovered that maintaining and promoting oral hygiene may be greatly enhanced by utilizing a unique kit having a combination of various dental compositions. Applicant has thus conceived of an oral hygiene kit capable of reducing dental plaque, suppressing bacterial growth, and reducing adhesiveness of plaque to thereby inhibit formation of dental caries.

Cavities are bacterial infections that cause the tooth structure to deteriorate and decay. The disclosure fights cavities using xylitol in several products and compositions explained in more detail below. Xylitol itself is a clinically proven cariostatic sweetener that reduces the ability of cavity-causing bacteria to impact oral health. Because of xylitol's cariostatic attributes, cavities cannot form in its presence. As such, it is a valuable tool in preventing and fighting cavities.

Strategies for treating dental caries have been varied in the past, but it is understood that to maintain good oral hygiene and reduce the occurrence of dental caries, oral mutans streptococci, or MS, must be suppressed. Using only a mouthwash, for example a mouthwash containing chlorhexidine, will inhibit MS levels for a short period of time, but oral MS levels tend to quickly return to baseline values without further intervention. It has been found that the regular use of products and compositions containing xylitol over a sustained period of time will further reduce the occurrence of dental caries by reducing MS levels.

According to Birkhed, D. in an article entitled “Cariologic aspects of xylitol and its use in chewing gum: a review,” found in Acta Odontol Scand 1994 April; 52(2):116-27, several studies have indicated that xylitol is not metabolized into acids either in pure cultures of oral microorganisms in vitro or in dental plaque in vivo. Consequently, persistent consumption of xylitol-sweetened chewing gum has resulted in reduction of dental plaque, suppression of mutans streptococci, and reduced adhesiveness of plaque. It has been demonstrated that a daily intake of two to three pieces of a xylitol chewing gum composition may result in a reduction of dental caries. Additionally, there are indications that regular and prolonged use of xylitol chewing gum may have a caries-preventive effect.

Additionally, several studies have resulted in further support of the use of xylitol as an effective way to treat DMFS as compared to fluoride. Xylitol has performed as well as or better than fluoride in side-by-side trials, as evidenced by the studies below.

A study by Scheinin A, Pienihakkinen K, Tiekso J, Banoczy J, Szoke J, Esztari I, Zimmermann P, Hadas E. entitled “Collaborative WHO xylitol field studies in Hungary. VII. Two-year caries incidence in 976 institutionalized children” and reported in Acta Odontol Scand. 1985 December; 43(6):381-7, assessed caries increment as influenced by partial substitution of sucrose by xylitol (X group) over a 2-year period in comparison with systemic fluoride (F group) and restorative treatment only (C group). The study consisted of 976 children (ages 6-12 years old). The 2-year DMFS increment was 3.8 in the X group, 4.8 in the F group, and 6.0 in the C group. The corresponding ratio (RS) between caries incidence and the tooth surface population at risk was RSX, 4.5; RSF, 5.5; and RSC, 7.5. The xylitol regimen was shown to result in a lower increment of caries than measured in the F and C groups. Accordingly, xylitol was shown to be an effective alternative to fluoride in treating DMFS.

Another study by Szoke J, Pienihakkinen K. Esztari I. Banoczy J. Scheinin A, entitled “Collaborative WHO xylitol field studies in Hungary. V. Three-year development of oral hygiene” and reported in Acta Odontol Scand. 1985 December; 43(6):371-6, illustrated the efficacy of xylitol in treating DMFS. In this study, the oral hygiene conditions were evaluated within a 3-year field study aimed at assessing the cariostatic value of partial substitution of sucrose by xylitol (X group) in comparison with systemic fluoride (F group) and restorative treatment only (C group). The observations were analyzed with regard to sex, age, experimental grouping, and total development. The oral hygiene conditions of the subjects were generally poor, and at the base-line examination only 26% of the children had acceptable oral hygiene. However, at the end of the study this level was increased by 16% and reached 42%. A definite improvement was measured only in the X group, in which the final values differed significantly (p less than 0.001) from the base-line values and also from the end situation in the F and C groups. It was thereby concluded and shown that the development was influenced by several factors, such as different snacking habits and access to sweets, the study per se, and xylitol-induced effects. Accordingly, xylitol was shown to be an effective alternative to fluoride in treating DMFS.

Xylitol can enhance the benefits of all prevention methods. Xylitol has been shown to be complementary with brushing, flossing, sealants, fluoride, and the antimicrobial rinse chlorhexidine. It should be noted that while high concentrations of fluoride can be damaging to humans because of its cumulative effects, lower concentrations of fluoride and chlorhexidine can be effective when teamed with xylitol.

However, the above publications and others do not disclose, teach or suggest the use of a complete oral hygiene system, kit and regimen for administering about 6 grams to about 10 grams of xylitol per day, as a daily dose, through multiple delivery mediums. Using the system, kit and regimen disclosed results in an effective amount of xylitol being administered to a patient or user over a sustained and prolonged period of time. The system, kit and regimen disclosed herein can be easily, effectively and continually followed by a patient or user, such that the desired anti-caries benefits may be obtained.

The disclosure is specifically designed to provide dental protection at the moments patients need it most. Specifically, the disclosure provides patients with protection when bacteria that causes cavities and tooth decay are most likely to be active, i.e. after meals and snacks.

The disclosure is directed to maintaining and promoting oral hygiene through the use of products and compositions containing xylitol. It will be appreciated that xylitol has a 5-carbon chemical structure that advantageously is not recognized by oral bacteria, including mutans streptococci. Additionally, xylitol is not fermented and, therefore, it will not result in acid production in plaque, and the pH levels in the mouth will remain neutral. Xylitol can also have the added benefit of actually reversing the fall of pH levels in the mouth (produced, for example, by sucrose) when, for instance, a person chews a xylitol-sweetened sugarless gum product. The oral pH is raised to a safe level, saliva flow is stimulated, which in turn helps to rinse away excess sucrose residues, and neutralizes any acids that have been formed.

Another benefit of xylitol is that saliva contains the minerals, in particular calcium and phosphate, which help to promote the remineralization of early cavities. The most fundamental difference between xylitol and other sweeteners is that xylitol reduces the amount of plaque and the virulence of mutans streptococci in plaque. Xylitol functions as a modulator of the oral flora, and when consumed regularly xylitol can even help to repair teeth by stimulating the remineralization of teeth already affected by cavities. Further, no matter how long xylitol is consumed (even during long term habitual use), oral bacteria will not adapt to metabolize xylitol, so the benefits of xylitol continue while it is being consumed, and has even been proved to continue after xylitol is no longer a part of daily use.

One illustrative embodiment of the disclosure may comprise a kit for promoting oral hygiene. It will be appreciated that the kit of the disclosure may comprise one individual product or composition, or the kit may comprise several products or compositions. For example, the disclosure may include a kit that comprises a chewing gum composition only, or the kit may include a combination of products or compositions, such as the chewing gum composition, a morsel composition, a paste-like composition, and an oral rinse composition. For exemplary purposes only and to streamline the disclosure, each of the products or compositions will be discussed below as being part of the kit. However, as mentioned above, the kit may comprise more or less products or compositions than the stated below.

The kit of the disclosure may include a chewing gum composition that may be chewed anytime, and particularly after meals and snacks. It will be appreciated that the chewing gum composition may be used as part of the kit or the chewing gum composition may be used alone, as an individually packaged product, without departing from the scope of the disclosure.

The chewing gum composition of the disclosure may be sweetened 100% by xylitol. In another embodiment, the chewing gum composition may comprise about 70% to about 100% xylitol by weight of sweeteners. Other sweeteners may be added to the chewing gum composition. The chewing gum composition may comprise an effective amount of xylitol by weight, and may further comprise gum base, at least one flavoring agent, glycerin, soy lecithin, gum arabic, titanium dioxide, carnauba wax, and resinous glaze. The chewing gum composition of the disclosure may be characterized by the absence of fluoride. However, it will be appreciated that the composition of the chewing gum may be modified to include, or may be used in conjunction with, fluoride without departing from the scope of the disclosure.

It will be appreciated that the amount of xylitol present in the chewing gum composition may be within a range from about 20% to about 85%, by weight, and more specifically within a range from about 25% to about 75%, by weight, inclusive. Applicant has found that an advantageous amount of xylitol present in the chewing gum composition may be in a range from about 30% to about 70% by weight, inclusive. Additional ranges that may supply a desired amount of xylitol may include a range from about 50% to about 85% by weight; or a range from about 60% to about 75% by weight; or a range from about 65% to about 70% by weight, inclusive. Other possible ranges that may supply a desired amount of xylitol may also include a range from about 20% to about 50% by weight; or a range from about 25% to about 40% by weight; or a range from about 30% to about 35% by weight, inclusive. It will be appreciated that any amount of xylitol may be present in the chewing gum composition in the ranges provided above, without departing from the spirit or scope of the disclosure.

It will be appreciated that the amount of xylitol present in the chewing gum composition may be modified depending upon the desired amount of xylitol to be administered to a user. For example, a directly compressible gum application may be used, and may be comprised of a greater amount of xylitol than a traditional chewing gum piece. In any event, administering at least about 0.5 to about 0.75 grams of xylitol per dose to a user through chewing the gum composition may be advantageous and may be expected to reduce MS levels, reduce dental plaque, suppress bacterial growth, and tends to reduce adhesiveness of plaque to thereby inhibit formation of, and reduce the incidence of, dental caries.

Administering about 0.5 grams to about 2.0 grams through the gum composition may be an effective method of administering xylitol at times when patient's need the cavity fighting protection most, which is after consumption of nutrition during meal and snacking time. Thus, xylitol may be present in a range of about 0.5 grams to about 2.0 grams, inclusive, in the gum composition without departing from the spirit or scope of the disclosure.

Illustratively, chewing the xylitol gum composition for at least five minutes after meals and snacks, when bacteria that causes dental caries is most active, is expected to be advantageous for reducing MS levels and for maintaining and promoting oral health.

### Example 1

### Xylitol Chewing Gum Composition

Xylitol Chewing Gum Ingredients:

| | |
|---|---|
| xylitol | 0.89 g |
| gum base | 0.32 g |
| natural flavors | 0.025 g |
| glycerin | less than 0.01 g |
| gum arabic | 0.01 g |
| tapioca dextrin | less than 0.01 g |
| soy lecithin | 0.02 g |
| titanium dioxide | 0.01 g |
| carnauba wax | 0.01 g |
| confectioners glaze | less than 0.01 g |

In the case of the exemplary Xylitol Chewing Gum of Example 1, the ingredients can be provided in the following proportions: xylitol—greater than about 60%; gum base greater than about 20%; natural flavors—less than about 2%; glycerin, gum arabic, and tapioca dextrin—less than about 1% each; lecithin—less than about 2%; and titanium dioxide, carnauba wax, and confectioner's glaze—less than about 1% each.

Another composition that may be present in the kit of the disclosure may be a morsel composition, such as mints, lozenges, or candies that may be consumed anytime, and particularly after meals and snacks. It will be appreciated that the morsel composition may be used as part of the kit or the morsel composition may be used alone, as an individually packaged product, without departing from the scope of the disclosure.

The morsel composition, such as mints, lozenges, or candies, may be sweetened 100% by xylitol. In another embodiment, the morsel composition may comprise about 70% to about 100% xylitol by weight of sweeteners. Other sweeteners may be added to the morsel composition. The morsel composition of the disclosure may further comprise an effective amount of xylitol, natural flavors, and calcium stearate. The morsel composition of the disclosure may be characterized by the absence of fluoride. However, it will be appreciated that the composition of the morsel may be modified to include, or may be used in conjunction with, fluoride without departing from the scope of the disclosure.

The amount of xylitol present in the morsel composition may be within a range from about 40% to about 100%, by weight, inclusive. More specifically, the amount of xylitol present in the morsel composition may be in a range from about 50% to about 99%, by weight, inclusive. For example, applicant has found that an advantageous amount of xylitol present in the morsel composition to be about 96% to about 98%, by weight. Additional ranges that may supply a desired amount of xylitol may include a range from about 75% to about 100% by weight; or a range from about 80% to about 99% by weight; or a range from about 85% to about 98% by weight; or a range from about 90% to about 96% by weight, inclusive. Other possible ranges that may supply a desired amount of xylitol may also include a range from about 40% to about 75% by weight; or a range from about 50% to about 70% by weight; or a range from about 60% to about 65% by weight, inclusive. It will be appreciated that the amount of xylitol present will likely depend upon the size of the morsel composition. It will be appreciated that any amount of xylitol may be used in the morsel composition as provided by the ranges above, without departing from the spirit or scope of the disclosure.

Applicant has found that administering at least about 0.5 grams of xylitol per dose through partaking of the morsel composition of the disclosure may reduce MS levels, may reduce dental plaque, may suppress bacterial growth, and may reduce adhesiveness of plaque to thereby inhibit formation of and reduce the incidence of, dental caries. It will be appreciated that in some instances it may be necessary to partake of at least one morsel composition, and in other instances it may be necessary to partake of more than one morsel composition to administer at least 0.075 g of xylitol in one dose, depending upon the size of the morsel composition. Applicant has found that collectively administering xylitol in the morsel composition in a range of about 0.5 grams to about 2.0 grams, inclusive, may be effective to reduce the occurrence of dental caries.

It will be appreciated that as used herein the term “partaking” may be defined as eating, sucking, chewing, swallowing, ingesting, devouring, consuming, or masticating a morsel composition. Specifically, partaking of the xylitol morsel composition for at least five minutes after meals and snacks, when bacteria that causes dental caries is most active, may be advantageous for reducing MS levels and for maintaining and promoting oral health.

### Example 2

### Xylitol Morsel Composition

Xylitol Morsel Ingredients:

| | |
|---|---|
| xylitol | 0.48 g |
| natural flavors | 0.01 g |
| calcium stearate | 0.01 g |

In the case of the exemplary Xylitol Morsel of Example 2, the ingredients can be provided in the following proportions: xylitol—more than about 95%; natural flavors—more than about 2%; and calcium stearate—more than about 2%.

Another composition that may be present in the kit of the disclosure may be a paste-like composition for brushing a user's teeth. It will be appreciated that the paste-like composition may be used as part of the kit or the paste-like composition may be used alone, as an individually packaged product, without departing from the scope of the disclosure.

The paste-like composition of the disclosure may comprise an effective amount of xylitol, water, silica, sorbitol, glycerin, at least one foaming agent, which may be sodium lauroyl sarcosinate or any other foaming agent that is currently known, or which may become known in the future, in the art, and at least one flavoring agent, for example mentha viridis (spearmint) leaf oil. The paste-like composition of the disclosure may be characterized by the absence of fluoride. However, it will be appreciated that the composition of the paste may be modified to include, or used in conjunction with, fluoride without departing from the scope of the disclosure.

It will be appreciated that the paste-like composition may comprise an amount of xylitol that may be present in a range from about 5% to about 50%, by weight, and more specifically in a range from about 15% to about 40%, by weight, inclusive. For example, applicant has found that an amount of xylitol present in the paste-like composition of about 25%, by weight, to be advantageous. Additional ranges that may supply a desired amount of xylitol may include a range from about 20% to about 50% by weight; or a range from about 25% to about 40% by weight; or a range from about 30% to about 35% by weight, inclusive. Other possible ranges that may supply a desired amount of xylitol may also include a range from about 5% to about 30% by weight; or a range from about 10% to about 25% by weight; or a range from about 15% to about 20% by weight, inclusive. However, it will be appreciated that one of skill in the art may modify the disclosure to include more or less than the above stated amount without departing from the scope of the disclosure. It will be appreciated that any amount of xylitol may be used in the paste-like composition as provided in the ranges above, without departing from the spirit or scope of the disclosure. It will be appreciated that the paste-like composition may comprise more than about 20% water and silica, by weight, inclusive. However, it will be appreciated that one of skill in the art may modify the disclosure to include more or less than the above stated amount without departing from the scope of the disclosure.

It will further be appreciated that the paste-like composition may comprise less than about 20% sorbitol, glycerin and sodium lauroyl sarcosinate, by weight. However, it will be appreciated that one of skill in the art may modify the disclosure to include more or less than the above stated amount without departing from the scope of the disclosure.

The paste-like composition of the disclosure may further comprise the following ingredients: stevia, cellulose gum, carrageenan, calcium glycerophosphate, copper PCA, and zinc PCA, each of which may be present in less than about 1%, by weight.

Applicant has found that brushing a user's teeth with the paste-like composition of the disclosure, which may comprise at least about 0.05 grams to about 0.75 grams of xylitol per dose, may be expected to reduce MS levels, reduce dental plaque, suppress bacterial growth, and may be expected to reduce adhesiveness of plaque to thereby inhibit formation of, and reduce the incidence of, dental caries. Specifically, brushing with the xylitol paste-like composition for at least one minute, sometime during the morning hours and sometime during the evening hours, or after partaking of the last meal and/or snack of the day, may be advantageous for reducing MS levels and for maintaining and promoting oral health.

Administering about 0.5 grams to about 2.0 grams through the paste-like composition may be an effective method of administering xylitol to provide a patient with needed cavity fighting protection. Thus, xylitol may be present in a range of about 0.5 grams to about 2.0 grams, inclusive, in the paste-like composition without departing from the spirit or scope of the disclosure.

### Example 3

### Xylitol Paste-Like Composition

Xylitol Paste Ingredients:

| | | |
|---|---|---|
| water | 28% | by weight |
| xylitol | 25% | by weight |
| silica | 23% | by weight |
| sorbitol | 13% | by weight |
| glycerin | 05% | by weight |
| sodium lauroyl sarcosinate | 03% | by weight |
| mentha viridis (spearmint) leaf oil | 01% | by weight |
| stevia | less than 01% | by weight |
| cellulose gum | less than 01% | by weight |
| carrageenan | less than 01% | by weight |
| calcium glycerophosphate | less than 01% | by weight |
| copper PCA | less than 01% | by weight |
| zinc PCA | less than 01% | by weight |

In the case of the exemplary Xylitol Paste of Example 3, the ingredients can be provided in the following proportions: water—greater than about 20%; silica—greater than about 20%; xylitol—about 20%; sorbitol—less than about 20%; glycerin—less than about 10%; sodium lauroyl sarcosinate—less than about 10%; mentha viridis (spearmint) leaf oil—less than about 3%; stevia—less than about 1%; cellulose gum—less than about 1%; carrageenan—less than about 1%; calcium glycerophosphate—less than about 1%; copper PCA—less than about 1%; and, zinc PCA—less than about 1%.

### Example 3A

### Xylitol Paste-Like Composition

Xylitol Paste Ingredients:

| | | |
|---|---|---|
| water | 28% | by weight |
| xylitol | 25% | by weight |
| calcium phosphate | 23% | by weight |
| sorbitol | 18% | by weight |
| sodium lauroyl sarcosinate | 03% | by weight |
| mentha viridis (spearmint) leaf oil | 01% | by weight |
| stevia | less than 01% | by weight |
| carrageenan | less than 01% | by weight |
| calcium glycerophosphate | less than 01% | by weight |
| copper PCA | less than 01% | by weight |
| zinc PCA | less than 01% | by weight |

In the case of the exemplary Xylitol Paste of Example 3A, the ingredients can be provided in the following proportions: water—greater than about 20%; calcium phosphate—greater than about 20%; xylitol—about 20%; sorbitol—less than about 20%; sodium lauroyl sarcosinate—less than about 10%; mentha viridis (spearmint) leaf oil—less than about 3%; stevia—less than about 1%; carrageenan—less than about 1%; calcium glycerophosphate—less than about 1%; copper PCA—less than about 1%; and, zinc PCA—less than about 1%.

Another composition that may be present in the kit of the disclosure may be an oral rinse composition that may be used after brushing the user's teeth. It will be appreciated that the oral rinse composition may be used as part of the kit or the oral rinse composition may be used alone, as an individually packaged product, without departing from the scope of the disclosure.

The oral rinse composition of the disclosure may comprise an effective amount of xylitol, water, glycerin, at least one flavoring agent, and sorbitol. The oral rinse composition of the disclosure may be characterized by the absence of fluoride. However, it will be appreciated that the composition of the oral rinse may be modified to include, or may be used in conjunction with, fluoride without departing from the scope of the disclosure.

It will be appreciated that the oral rinse composition may comprise more than about 20% water, by weight. More specifically, water may present in the oral rinse composition in a range from about 20% to about 80%, by weight, inclusive. Other possible ranges include a range from about 50% to about 80% by weight; or a range from about 60% to about 75% by weight; or a range from about 65% to about 70% by weight, inclusive. Additional ranges may also include a range from about 20% to about 50% by weight; or a range from about 25% to about 45% by weight; or about 30% to about 35% by weight, inclusive. However, it will be appreciated that one of skill in the art may modify the disclosure to include more or less than the above stated amount without departing from the scope of the disclosure. Further, xylitol may be present in the oral rinse composition in a range of about 0.5 grams to about 1.5 grams, inclusive, in the gum composition without departing from the spirit or scope of the disclosure.

It will be appreciated that the oral rinse composition may comprise an amount of xylitol that may be present in a range from about 5% to about 60%, by weight, and more specifically in a range from about 15% to about 50%, by weight, inclusive. For example, applicant has found that including about 25% by weight of xylitol in the oral rinse composition to be an advantageous amount of xylitol. Additional ranges that may supply a desired amount of xylitol may include a range from about 40% to about 60% by weight; or a range from about 45% to about 65% by weight; or a range from about 50% to about 65% by weight, inclusive. Other possible ranges that may supply a desired amount of xylitol may also include a range from about 5% to about 40% by weight; or a range from about 15% to about 30% by weight; or a range from about 20% to about 25% by weight, inclusive. However, it will be appreciated that one of skill in the art may modify the disclosure to include more or less than the above stated amounts without departing from the scope of the disclosure. It will be appreciated that any amount of xylitol may be used in the oral rinse composition as provided by the ranges above, without departing from the spirit or scope of the disclosure.

Additionally, the oral rinse composition may comprise less than about 20% glycerin, by weight, specifically glycerin may be present in the oral rinse composition in a range from about 5% to about 20%, by weight, inclusive. Other possible ranges may include a range from about 3% to about 10% by weight; or a range from about 5% to about 8% by weight. Additional ranges may also include a range from about 10% to about 20% by weight; or a range from about 12% to about 18% by weight; or about 15% to about 17% by weight, inclusive. However, it will be appreciated that one of skill in the art may modify the disclosure to include more or less than the above stated amount without departing from the scope of the disclosure. The oral rinse composition may further comprise polysorbate 20, mentha viridis leaf oil, sodium benzoate, menthol, citric acid, zinc PCA, and ascorbic acid, which may be present in the oral rinse composition in less than about 1%, by weight.

Applicant has found that rinsing a user's teeth and oral cavity with the oral rinse composition of the disclosure, which may comprise at least about 0.5 grams to about 0.75 grams of xylitol per dose, may be expected to reduce MS levels, expected to reduce dental plaque, may be expected to suppress bacterial growth, and may also be expected to reduce adhesiveness of plaque to thereby inhibit formation of, and reduce the incidence of, dental caries. Specifically, rinsing with the xylitol oral rinse composition for at least one minute after brushing, and at least in the evening, may be advantageous for reducing MS levels and for maintaining and promoting oral health.

### Example 4

### Xylitol Oral Rinse Composition

Xylitol Oral Rinse Ingredients:

| | | |
|---|---|---|
| water | 68% | by weight |
| xylitol | 25% | by weight |
| glycerin | 05% | by weight |
| sorbitol | 01% | by weight |
| polysorbate 20 | less than 01% | by weight |
| mentha viridis leaf oil | less than 01% | by weight |
| sodium benzoate | less than 01% | by weight |
| menthol | less than 01% | by weight |
| citric acid | less than 01% | by weight |
| zinc PCA | less than 01% | by weight |
| ascorbic acid | less than 01% | by weight |

In the case of the exemplary Xylitol Oral Rinse of Example 4, the ingredients can be provided in the following proportions: water—greater than about 50%; xylitol—greater than about 20%; glycerin—less than about 10%; sorbitol—less than about 3%; polysorbate 20—less than about 1%; mentha viridis leaf oil—less than about 1%; sodium benzoate—less than about 1%; menthol—less than about 1%; citric acid—less than about 1%; zinc PCA—less than about 1%; and, ascorbic acid—less than about 1%.

### Example 4A

### Xylitol Oral Rinse Composition

Xylitol Oral Rinse Ingredients:

| | | |
|---|---|---|
| water | 68% | by weight |
| xylitol | 25% | by weight |
| glycerin | 05% | by weight |
| sorbitol | 01% | by weight |
| PEG-40 hydrogenated castor oil | less than 01% | by weight |
| mentha viridis leaf oil | less than 01% | by weight |
| potassium sorbate | less than 01% | by weight |
| menthol | less than 01% | by weight |
| malic acid | less than 01% | by weight |
| zinc PCA | less than 01% | by weight |
| ascorbic acid | less than 01% | by weight |

In the case of the exemplary Xylitol Oral Rinse of Example 4A, the ingredients can be provided in the following proportions: water—greater than about 50%; xylitol—greater than about 20%; glycerin—less than about 10%; sorbitol—less than about 3%; PEG-40 hydrogenated castor oil—less than about 1%; mentha viridis leaf oil—less than about 1%; potassium sorbate—less than about 1%; menthol—less than about 1%; malic acid—less than about 1%; zinc PCA—less than about 1%; and, ascorbic acid—less than about 1%.

It should be noted that other ingredients could be used in the compositions of either Example 4 or Example 4A instead of sodium benzoate as illustrated above without departing from the spirit or scope of the disclosure. For example, sodium benzoate could be replaced with potassium sorbate, benzoate potassium, or benzoic acid without departing from the spirit or scope of the disclosure.

It should further be noted that other ingredients could be used in Example 4 or Example 4A instead of citric acid as illustrated above without departing from the spirit or scope of the disclosure. For example, citric acid could be replaced with malic acid or tartaric acid without departing from the spirit or scope of the disclosure.

Likewise, it should be noted that other ingredients having similar characteristics and properties as the above-identified exemplary ingredients in the chewing gum composition, the morsel composition, the paste-like composition, and the oral rinse composition may be used and substituted in the disclosure, without departing from the spirit or scope of the disclosure.

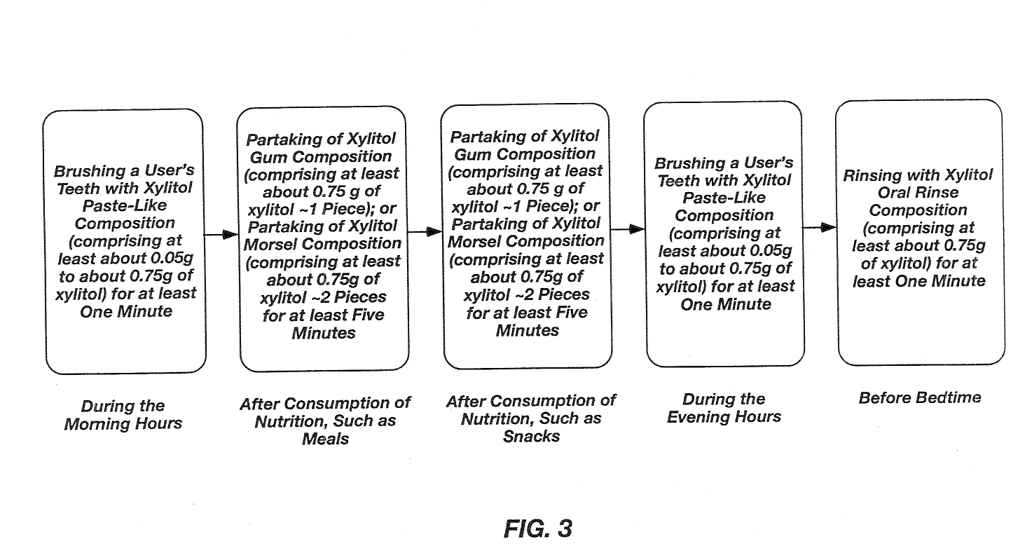

Referring now to FIGS. 3-6, which illustrate exemplary methods of using the compositions described in detail above. More specifically, FIG. 3 illustrates a method for promoting oral hygiene and to reduce dental plaque, suppress bacterial growth, and reduce adhesiveness of plaque in a user's oral cavity to thereby inhibit formation of dental caries. The steps illustrated in FIG. 3 include the following. First, during the morning hours of the day a user may brush his or her teeth using the xylitol paste-like composition, which may advantageously comprise at least about 0.05 g to about 0.75 g of xylitol per dose or per use. Applicant has found that brushing with the xylitol paste-like composition for at least one minute allows the xylitol present in the paste-like composition to provide its beneficial effects.

The second and third steps of the method include the user partaking of the xylitol gum composition, which may advantageously comprise at least about 0.75 g of xylitol in one piece of the gum composition, after consumption of nutrition, such as meals or snacks when tooth brushing is not as convenient or readily available. Alternatively, the user may select to partake of the xylitol morsel, which may advantageously comprise at least about 0.75 g of xylitol in one or more pieces of the morsel composition, instead of, or in addition to, partaking of the chewing gum composition. Applicant has found that partaking of the xylitol chewing gum or morsel compositions, or any combination thereof, for at least five minutes allows the xylitol present in those compositions to provide its beneficial effects.

The fourth and fifth steps of the method illustrated in FIG. 3 may be grouped together for convenience of the user, although such is not required. The fourth step of the method includes the user brushing his or her teeth using the xylitol paste-like composition, which may advantageously comprise at least about 0.05 g to about 0.75 g of xylitol per dose or per use, during the evening hours. Typically, this step will be after the last meal or snack of the day and just prior to bedtime. Thereafter, the fifth step of the method may be completed. The fifth step of the method includes rinsing the user's teeth and oral cavity with the oral rinse composition, which may advantageously comprise at least about 0.75 g of xylitol per dose or per use, before bedtime. Applicant has found that brushing with the xylitol paste-like composition for at least one minute; and rinsing the user's teeth and oral cavity with the xylitol oral rinse composition for at least one minute allows the xylitol present in the paste-like and oral rinse compositions to provide its beneficial effects.

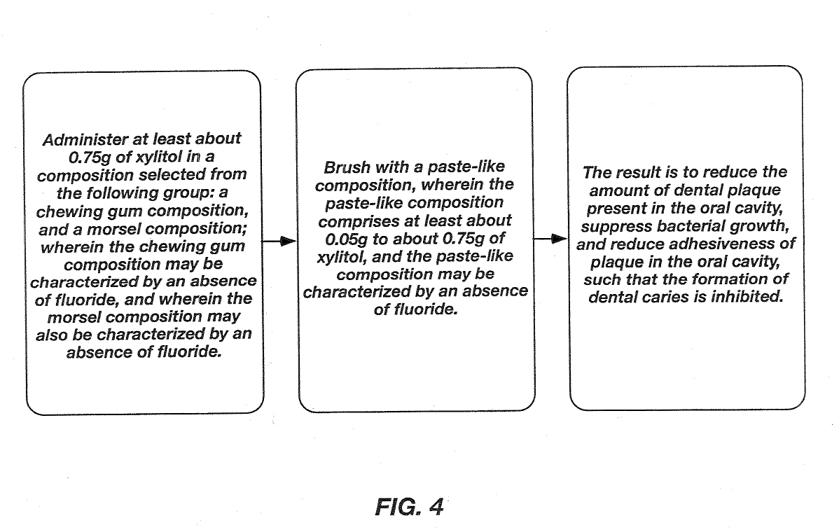

It will be appreciated that there may be other methods that may be used without departing from the spirit and scope of the disclosure. For example, a combination of the above enumerated steps comprising more or less than the above referenced steps may be beneficial, as long as the effective amount of xylitol may be supplied to the user's teeth and oral cavity. FIGS. 4-6 illustrate such examples, but it should be noted that there may be other combinations that may also be utilized without departing from the scope of the disclosure.

FIG. 4 illustrates another exemplary method for promoting oral hygiene and to reduce dental plaque, suppress bacterial growth, and reduce adhesiveness of plaque in a user's oral cavity to thereby inhibit formation of dental caries is illustrated. The steps of the method illustrated in FIG. 4 may comprise, first, administering at least about 0.75 g of xylitol in either a chewing gum composition or a morsel composition to a user, wherein both the chewing gum composition and the morsel composition may be characterized by an absence of fluoride. Second, brushing the user's teeth with a paste-like composition, wherein the paste-like composition may comprise at least about 0.05 g to about 0.75 g of xylitol, and may be characterized by an absence of fluoride. Following the above steps may reduce dental plaque, suppress bacterial growth, and reduce adhesiveness of plaque, such that the formation of dental caries is inhibited.

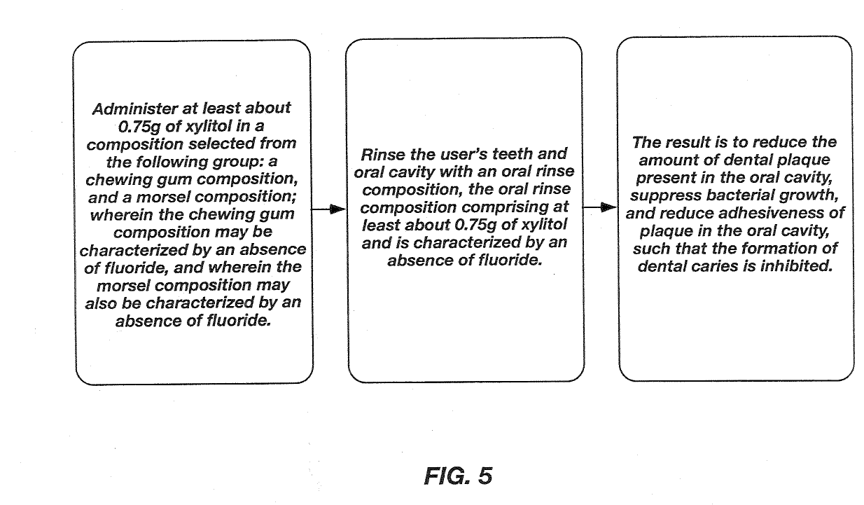

Referring now to FIG. 5, wherein another exemplary method for promoting oral hygiene and to reduce dental plaque, suppress bacterial growth, and reduce adhesiveness of plaque in a user's oral cavity to thereby inhibit formation of dental caries is illustrated. The steps of the method illustrated in FIG. 5 may comprise, first, administering at least about 0.75 g of xylitol in either a chewing gum composition or a morsel composition, wherein both the chewing gum composition and the morsel composition may be characterized by an absence of fluoride. Second, rinsing the user's teeth and oral cavity with an oral rinse composition that may comprise at least about 0.75 g of xylitol and may be characterized by an absence of fluoride. Following the above steps may reduce dental plaque, suppress bacterial growth, and reduce adhesiveness of plaque to thereby inhibit formation of dental caries.

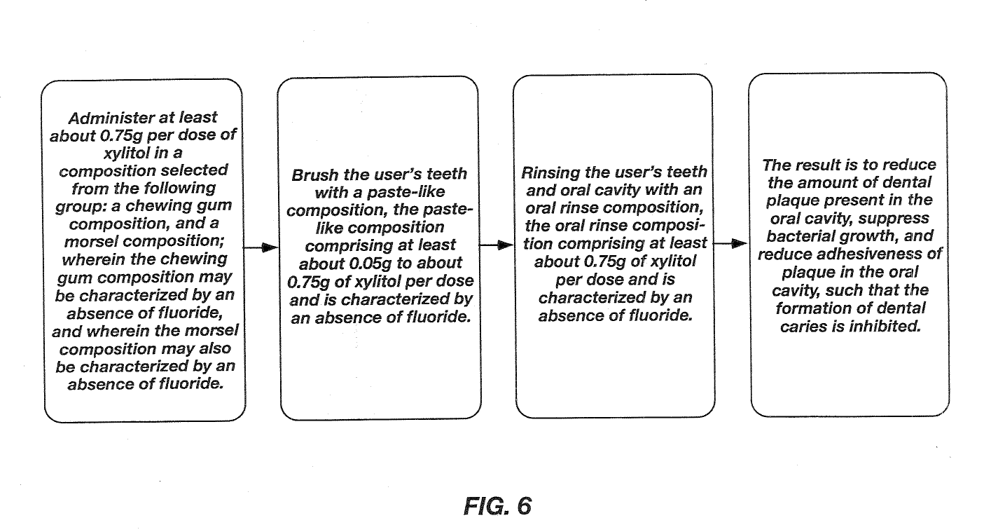

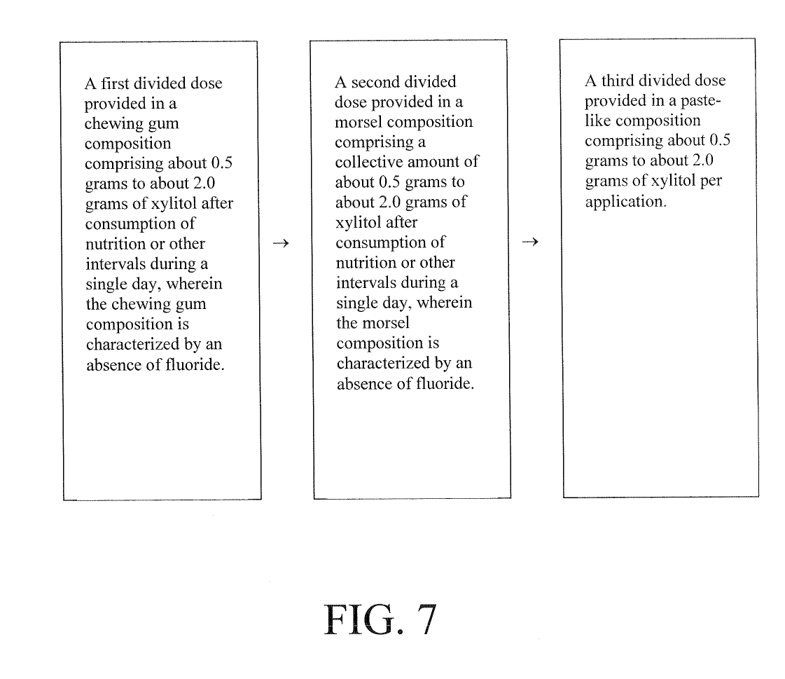

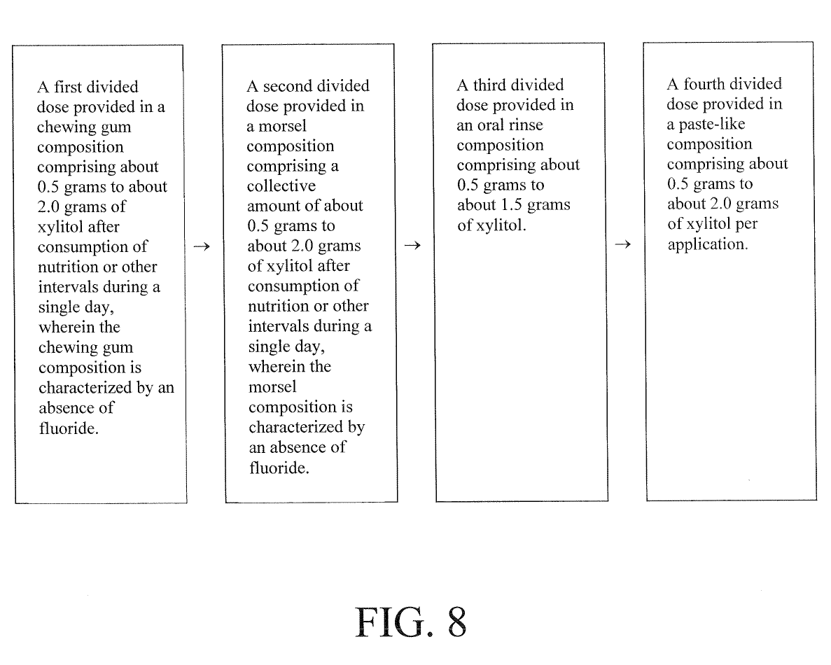

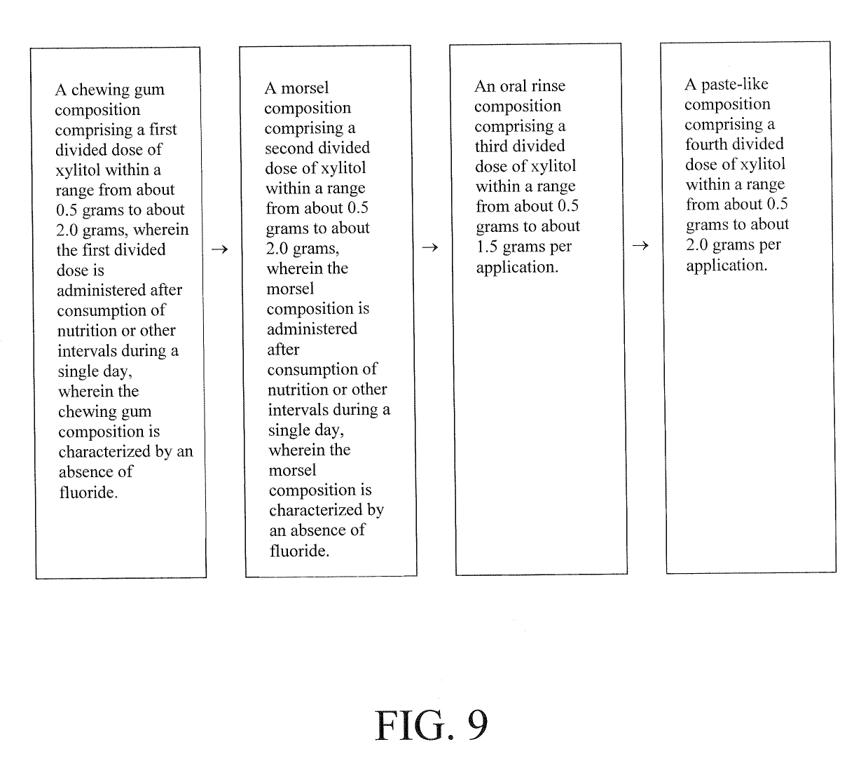

Referring now to FIG. 6, another method for promoting oral hygiene and to reduce dental plaque, suppress bacterial growth, and reduce adhesiveness of plaque in a user's oral cavity to thereby inhibit formation of dental caries is illustrated. The steps of the method may include, first, administering at least about 0.75 g of xylitol in either a chewing gum composition or a morsel composition to a user, wherein both the chewing gum composition and the morsel composition may be characterized by an absence of fluoride. Second, brushing the user's teeth with a paste-like composition, wherein the paste-like composition may comprise at least about 0.05 g to about 0.75 g of xylitol, and may be characterized by an absence of fluoride. Last, rinsing the user's teeth and oral cavity with an oral rinse composition that may comprise at least about 0.75 g of xylitol and may be characterized by an absence of fluoride. Following the above steps may reduce dental plaque, suppress bacterial growth, and reduce adhesiveness of plaque to thereby inhibit formation of dental caries.

The compositions of the disclosure can be used for promoting oral hygiene, reducing dental plaque, suppressing bacterial growth, and reducing adhesiveness of plaque in a user's oral cavity to thereby inhibit formation of dental caries by orally administering an effective amount of the compositions. It will be appreciated that an effective amount of xylitol may be provided through use of one or more of the compositions disclosed. A daily effective amount of xylitol may be in the range of about 6 grams to about 10 grams, inclusive. It is to be understood that daily effective amounts or doses of xylitol can be divided, wherein two or more administrations of divided doses are used to deliver a complete daily dose. Multiple doses of the same composition can also be administered, but it is recommended that daily consumption of xylitol no matter what combination of compositions disclosed herein are used together fall within the range of about 6 grams to about 10 grams of xylitol, inclusive. Thus, a complete daily dose of xylitol may be administered to a user through the mediums of the gum composition, the morsel composition, the paste-like composition and the oral rinse composition to obtain the desired anti-caries benefits.

In accordance with the features and combinations described above, a useful regimen or method for promoting oral hygiene and to reduce dental plaque, suppress bacterial growth, and reduce adhesiveness of plaque in a user's oral cavity to thereby inhibit formation of dental caries, may include the steps of:

(a) brushing a user's teeth with a paste-like composition during morning hours, the paste-like composition comprising an amount of xylitol between a range of about 0.05 g to about 0.75 g and is characterized by an absence of fluoride;

(b) administering at least about 0.75 g of xylitol after meals or snacks in a composition selected from the group consisting of: a chewing gum composition, and a morsel composition, wherein the chewing gum composition is characterized by an absence of fluoride, and wherein the morsel composition is characterized by an absence of fluoride;

(c) brushing the user's teeth with the paste-like composition during evening hours; and

(d) rinsing the user's oral cavity with an oral rinse comprising at least about 0.75 g of xylitol before bedtime, to reduce dental plaque, suppress bacterial growth, and reduce adhesiveness of plaque to thereby inhibit formation of dental caries.

It will be appreciated that other products and compositions may be utilized in accordance with the disclosure, including: xylitol-coated floss, cavity protection gel for infants and toddlers who are teething, a xylitol tooth varnish, a xylitol hard candy, a xylitol gummy bear and other xylitol confectionery products.

Those having ordinary skill in the relevant art will appreciate the advantages provided by the features of the disclosure. For example, it is a potential feature of the disclosure to provide a kit for promoting oral health and hygiene that is simple to use, and that may comprise a chewing gum composition, a morsel composition, a paste-like composition, and an oral rinse composition. Another potential feature of the disclosure is to provide such a kit for promoting oral health and hygiene, wherein each of the compositions of the kit comprises effective amounts of xylitol.

It is a further potential feature of the disclosure to provide the components of the kit for promoting oral health and hygiene individually, such that an amount of a single composition may be purchased individually without each of the other compositions. For example, a xylitol chewing gum composition, a xylitol morsel composition, i.e. mints, lozenges and candies, a xylitol paste-like composition, a xylitol oral rinse composition, as well as other oral products containing xylitol may be provided for individual distribution, i.e. through retail or wholesale outlets. It is another potential feature to promote oral health and hygiene through use of a kit that may comprise products that are appetizing and that reduce dental plaque, suppress bacterial growth, and reduce adhesiveness of plaque to thereby inhibit formation of dental caries.

In the foregoing Detailed Description, various features of the disclosure are grouped together in a single embodiment for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing, disclosed embodiment. Thus, the following claims are hereby incorporated into this Detailed Description by this reference, with each claim standing on its own as a separate embodiment of the disclosure.

It is to be understood that the above-described compositions and arrangements are only illustrative of the application of the principles of the disclosure. Numerous modifications and alternative arrangements may be devised by those skilled in the art without departing from the spirit and scope of the disclosure and the appended claims are intended to cover such modifications and arrangements. Thus, while the disclosure has been described above with particularity and detail, it will be apparent to those of ordinary skill in the art that numerous modifications, including, but not limited to, variations in amounts, ingredients, compositions, configurations, process steps, materials, form, function and manner of use, may be made without departing from the principles and concepts set forth herein.

1-117. (canceled)

118. A method for promoting oral hygiene comprising:
administering an effective amount of xylitol during a single day to operate as an antimicrobial agent for reducing dental plaque, suppressing bacterial growth, and reducing adhesiveness of plaque in a user's oral cavity to thereby inhibit formation of dental caries;
administering the effective amount of xylitol through different delivery mediums comprising:
(a) a first divided dose provided in a chewing gum composition comprising about 0.5 grams to about 2.0 grams of xylitol after consumption of nutrition or other intervals during a single day, wherein the chewing gum composition is characterized by an absence of fluoride;
(b) a second divided dose provided in a morsel composition comprising a collective amount of about 0.5 grams to about 2.0 grams of xylitol after consumption of nutrition or other intervals during a single day, wherein the morsel composition is characterized by an absence of fluoride; and
(c) a third divided dose provided in a paste-like composition comprising about 0.5 grams to about 2.0 grams of xylitol per application;
wherein the first divided dose, the second divided dose and the third divided dose equal a daily dose of xylitol.

119. The method of claim 118, wherein the method further comprises administering a fourth divided dose of an oral rinse composition comprising about 15% to about 25% by weight xylitol, such that about 0.5 grams to about 1.5 grams of xylitol is administered to a user.

120. The method of claim 118, wherein the daily dose of xylitol is equal to about 6 grams to about 10 grams.

121. The method of claim 118, wherein administering the first divided dose further comprises chewing the chewing gum composition after consumption of nutrition for at least five minutes, such that a user's oral cavity is exposed to the xylitol present in the chewing gum composition.

122. The method of claim 118, wherein administering the second divided dose further comprises partaking of the morsel composition after consumption of nutrition for a sufficient time, such that a user's oral cavity is exposed to the xylitol present in the morsel composition.

123. The method of claim 118, wherein administering the third divided dose further comprises brushing a user's teeth for at least one minute with the paste-like composition, such that the user's oral cavity is exposed to the xylitol present in the paste-like composition.

124. The method of claim 119, wherein a user's oral cavity is exposed to:
about 2 grams to about 4 grams of xylitol per day from the first divided dose;
about 1 grams to about 4 grams of xylitol per day from the second divided dose;
about 2 grams to about 3 grams of xylitol per day from the third divided dose; and
about 1 gram to about 2 grams of xylitol per day from the fourth divided dose;
wherein the user's oral cavity is exposed to about 6 gram to about 10 grams of xylitol per day incrementally over the course of the day.

125. The method of claim 118, wherein the chewing gum composition comprises about 70% to about 100% xylitol by weight of sweeteners, and further comprises gum base, at least one flavoring agent, glycerin, soy lecithin, gum arabic, titanium dioxide, carnauba wax, and resinous glaze.

126. The method of claim 118, wherein the morsel composition comprises about 70% to about 100% xylitol by weight of sweeteners, and further comprises natural flavors, and calcium stearate.

127. The method of claim 118, wherein the paste-like composition comprises an effective amount of water, silica, sorbitol, glycerin, a foaming agent, and at least one flavoring agent.

128. The method of claim 118, wherein xylitol is present in the paste-like composition within a range from about 5% to about 50%, by weight.

129. The method of claim 118, wherein xylitol is present in the chewing gum composition within a range from about 20% to about 80%, by weight.

130. The method of claim 118, wherein the chewing gum composition comprises greater than about 60% xylitol by weight.

131. The method of claim 130, wherein the chewing gum composition comprises greater than about 20% gum base by weight, less than about 2% natural flavors and lecithin by weight, and less than about 1% glycerin, gum arabic, tapioca dextrin, titanium dioxide, carnauba wax, and confectioner's glaze by weight.

132. The method of claim 118, wherein xylitol is present in the morsel composition in a range from about 40% to about 100%, by weight.

133. The method of claim 132, wherein the morsel composition comprises greater than about 2% natural flavors and calcium stearate by weight.

134. The method for promoting oral hygiene of claim 118, wherein the paste-like composition comprises more than about 20% water, xylitol and silica by weight and less than about 20% sorbitol, glycerin, and sodium lauroyl sarcosinate by weight; wherein the paste-like composition comprises less than about 1% of stevia, cellulose gum, carrageenan, calcium glycerophosphate, copper PCA, and zinc PCA.

135. The method of claim 119, wherein xylitol is present in the oral rinse composition in a range from about 5% to about 60%, by weight, wherein water is present in the oral rinse composition in a range from about 20% to about 80%, by weight, wherein glycerin is present in the oral rinse composition in a range from about 5% to about 20%, by weight, wherein the oral rinse composition further comprises at least one flavoring agent, and sorbitol; wherein the oral rinse composition further comprises a first ingredient selected from the group consisting of: polysorbate 20 and PEG-40 hydrogenated castor oil; and further comprises a second ingredient selected from the group consisting of: sodium benzoate, potassium sorbate, benzoate potassium, and benzoic acid; and further comprises a third ingredient selected from the group consisting of: citric acid, malic acid and tartaric acid; wherein the oral rinse composition comprises less than 1% by weight of the first ingredient, the second ingredient and the third ingredient.

136. A method for promoting oral hygiene, comprising:
administering an effective amount of xylitol during a single day to operate as an antimicrobial agent for reducing dental plaque, suppressing bacterial growth, and reducing adhesiveness of plaque in a user's oral cavity to thereby inhibit formation of dental caries;
administering the effective amount of xylitol through different delivery mediums comprising:
(a) a first divided dose provided in a chewing gum composition comprising about 0.5 grams to about 2.0 grams of xylitol after consumption of nutrition or other intervals during a single day, wherein the chewing gum composition is characterized by an absence of fluoride;
(b) a second divided dose provided in a morsel composition comprising a collective amount of about 0.5 grams to about 2.0 grams of xylitol after consumption of nutrition or other intervals during a single day, wherein the morsel composition is characterized by an absence of fluoride;
(c) a third divided dose provided in an oral rinse composition comprising about 0.5 grams to about 1.5 grams of xylitol; and
(d) a fourth divided dose provided in a paste-like composition comprising about 0.5 grams to about 2.0 grams of xylitol per application;
wherein the first divided dose, the second divided dose, the third divided dose and the fourth divided dose equal a daily dose of xylitol.

137. The method of claim 136, wherein the daily dose of xylitol is equal to about 6 grams to about 10 grams of xylitol.

138. A kit for promoting oral hygiene by administering an effective amount of xylitol during a single day to operate as an antimicrobial agent for reducing dental plaque, suppressing bacterial growth, and reducing adhesiveness of plaque in a user's oral cavity to thereby inhibit formation of dental caries, the kit comprising:
a plurality of different delivery mediums, wherein each delivery medium comprises a divided dose of xylitol that collectively delivers a complete daily dose of xylitol to a user, wherein the plurality of different delivery mediums comprises:
(a) a chewing gum composition comprising a first divided dose of xylitol within a range from about 0.5 grams to about 2.0 grams, wherein the first divided dose is administered after consumption of nutrition or other intervals during a single day, wherein the chewing gum composition is characterized by an absence of fluoride;
(b) a morsel composition comprising a second divided dose of xylitol within a range from about 0.5 grams to about 2.0 grams, wherein the morsel composition is administered after consumption of nutrition or other intervals during a single day, wherein the morsel composition is characterized by an absence of fluoride;
(c) an oral rinse composition comprising a third divided dose of xylitol within a range from about 0.5 grams to about 1.5 grams per application; and
(d) a paste-like composition comprising a fourth divided dose of xylitol within a range from about 0.5 grams to about 2.0 grams per application;
wherein the first divided dose, the second divided dose, the third divided dose and the fourth divided dose equal a daily dose of xylitol.

139. The kit of claim 138, wherein the chewing gum composition comprises about 70% to about 100% xylitol by weight of sweeteners, and comprises gum base, at least one flavoring agent, glycerin, lecithin, gum arabic, titanium dioxide, carnauba wax, and resinous glaze;
wherein xylitol is present in the chewing gum composition in a range from about 20% to about 85%, by weight;
wherein the morsel composition comprises about 70% to about 100% xylitol by weight of sweeteners and comprises natural flavors and calcium stearate;
wherein the paste-like composition comprises water, silica, sorbitol, glycerin, sodium lauroyl sarcosinate, and at least one flavoring agent;
wherein xylitol is present in the paste-like composition in a range from about 5% to about 50%, by weight;
wherein the paste-like composition comprises more than about 20% silica, by weight;
wherein the paste-like composition comprises less than about 20% sorbitol, by weight;
wherein the paste-like composition further comprises less than about 1%, by weight, of stevia, cellulose gum, carrageenan, calcium glycerophosphate, copper PCA, and zinc PCA; and
wherein the oral rinse composition comprises water, glycerin, at least one flavoring agent, and sorbitol;
wherein the oral rinse composition further comprises less than about 1%, by weight of polysorbate 20, mentha viridis leaf oil, sodium benzoate, menthol, citric acid, zinc PCA, and ascorbic acid;
wherein the oral rinse composition comprises more than about 20% water, by weight;
wherein xylitol is present in the oral rinse composition in a range from about 5% to about 60%, by weight; and
wherein the oral rinse composition comprises less than about 10% glycerin, by weight.

140. A kit for promoting oral hygiene and reducing dental plaque, suppress bacterial growth, and reduce adhesiveness of plaque in a user's oral cavity to thereby inhibit formation of dental caries, the kit comprising:
a chewing gum composition to be used after meals or snacks, wherein the chewing gum composition is sweetened substantially by xylitol, wherein the chewing gum composition comprises an effective amount of xylitol, and wherein the chewing gum composition is characterized by the absence of fluoride;
a morsel composition sweetened substantially by xylitol, wherein the morsel composition comprises an effective amount of xylitol, natural flavors, and calcium stearate, and wherein the morsel composition is characterized by the absence of fluoride; and
an oral rinse composition comprising an effective amount of xylitol, water, glycerin, at least one flavoring agent, and sorbitol, wherein the oral rinse is used after brushing a user's teeth; and
a paste-like composition for brushing a user's teeth, the paste-like composition comprising an effective amount of xylitol and water;
wherein the chewing gum composition when used in a regimen in conjunction with the morsel composition, the oral rinse composition, and the paste-like composition reduces dental plaque, suppresses bacterial growth, and reduces adhesiveness of plaque in a user's oral cavity, to thereby inhibit formation of dental caries.
